(19) United States
(12) Patent Application Publication
(10) Pub. No.: US 2011/0071497 A1
(43) Pub. Date: Mar. 24, 2011

(54) BALLOON FIXATED CATHETER

(76) Inventors: Red M. Alinsod, Laguna Beach, CA (US); James Christensen, Glendora, CA (US)

(21) Appl. No.: 12/565,633

(22) Filed: Sep. 23, 2009

(51) Int. Cl. A61M 25/04; A61M 5/142

(52) U.S. Cl. 604/509; 604/96.01; 604/151

(57) ABSTRACT

A balloon catheter for the delivery of a medication comprises a balloon fixation portion and a medication infusion portion. The balloon fixation portion comprises an expandable balloon at the distal end of a tube, the balloon inflatable to a size sufficient to retain the catheter within the area of placement, and a check valve at the proximal end of the balloon fixation portion for inflation or deflation of the balloon. The medication infusion portion comprises a tube with a precise diameter lumen functioning to control the flow rate therethrough, the tubing having one or more holes along its distal portion for infusion of medication into the patient and means on its proximal end for attachment to an infusion pump. The balloon fixation portion and the medication infusion portion are independently produced and then joined along a distal portion of both to provide a unitary catheter for placement into the body.

### FIELD OF THE INVENTION

The present invention relates to a catheter which can be used for the infusion of a medication into the body, and more particularly, to a balloon fixated catheter comprised of a first portion containing a balloon which can be inflated to temporarily fixate the catheter in the body, and a second portion used to infuse the medication into the body, both portions being joined in a side-by-side manner along their distal portion.

### BACKGROUND OF THE INVENTION

Catheters used for infusion of an anesthetic into the body for the relief of pain are well known in the prior art. These catheters generally consist of a hollow, flexible tube placed through the skin and into some portion of the body. The proximal end of the catheter is connected to a fluid source containing the medication to be infused. The distal end of the catheter usually contains one or more exit holes through which the medication flows into the body. Generally, the soft tissues of the body are used to hold these catheters in place during the period of infusion. In addition, an adhesive dressing is usually applied over the catheter where it exits the skin in order to stabilize the catheter further and prevent pull-out and infection.

This method of holding a catheter in place is often not very secure and the distal end of these catheters can easily be pulled out of the body or migrate further into the body. In some cases, sutures are placed around the distal end of the catheter to stabilize them. If these sutures are not placed correctly, they can collapse or crimp the catheter. During removal of the catheter either surgical intervention is required to remove the sutures or the catheter is pulled out and the sutures are left in place.

Where there is insufficient soft tissue to hold a catheter in place, such as when a catheter is inserted into a vein or artery, various styles of tapes or adhesive backed dressings are used to secure the catheter to the patient's skin. Alternatively, various styles of suturing tabs are disclosed in patents, such as U.S. Pat. No. 4,962,757 to Stefan, which can be wrapped around a catheter to enable the catheter to be more securely attached to the patient using sutures. While these methods of securing a catheter are satisfactory for preventing catheter pull-out, they are not suitable for stabilizing the portion of the catheter placed inside the patient. Unless a catheter is placed inside a vein or artery, the distal end of the catheter could be free to move around and could migrate from the position where it was initially placed.

In cases where a catheter is used to infuse an anesthetic into a body cavity, there may be insufficient soft tissue to hold the catheter in place. In these cases a catheter that can be anchored to the infusion site is desirable. U.S. Pat. No. 6,482,184 to Christensen, et al, describes a catheter with a filamentous attachment means that can be used to temporarily attach a catheter at the infusion site. A suture is placed at the infusion site. The ends of this suture are then tied through the filamentous loop of the attachable catheter. The filamentous loop is pulled through the attachment tube of the catheter while the catheter is slid up over the suture. When the distal end of the attachable catheter is at the infusion site, the suture, which has now been pulled through the catheter attachment tube, is cut from the filamentous loop and tied around the attachable catheter to secure the catheter in place. This fixates the distal end of the catheter at the infusion site in the patient's body. After infusion is complete, the suture is cut and the catheter and suture are removed from the body. This technology works well for easy to reach infusion sites such as those associated with hemorrhoids, etc. However, for deeper sites, it is cumbersome to place the suture and to tie the suture around the filamentous loop.

Several styles of catheters have been described in the prior art literature which utilize a balloon to retain the catheter in the body during infusion or withdrawal of a medication from the body. Foley style balloon catheters are fixated in the bladder using a balloon in order to drain the bladder. They consist of a multilumen coaxial tube containing a balloon inflation lumen and a bladder drainage lumen. A balloon is fixed to the distal end of the inflation lumen which can be inflated through a check valve assembly contained at the proximal end of the inflation lumen. The distal end of the drainage lumen contains a hole for entry of urine and the proximal end contains a connector to attach the catheter to a collection bag.

Similarly, trans-cervical catheters, used to administer saline to the uterus for diagnostic purposes, utilize a balloon to fixate the catheter distal end in the uterus. They also consist of a multilumen coaxial tube containing a balloon inflation lumen and a saline infusion lumen. A balloon is fixed to the distal end of the inflation lumen which can be inflated through a check valve assembly contained at the proximal end of the inflation lumen. The distal end of the infusion lumen contains a hole for infusion of saline and the proximal end contains a connector to attach the catheter to a syringe containing saline.

Both of these styles of balloon fixated catheters utilize a multilumen, coaxial tube between their distal and proximal ends. While this may be cost effective for these styles of catheters, it is very difficult to achieve a highly accurate and consistent lumen internal diameter which is required if the lumen is to be used as a flow restrictor. Further, catheters having different flow rates would require different, highly accurate and consistent lumen internal diameters.

### SUMMARY OF THE INVENTION

The present invention discloses a balloon catheter that can be easily and reliably fixated in the body in order to provide infusion of a medication for the relief of pain in the patient at or near the fixation site. The balloon catheter consists of a balloon fixation portion intended to hold the catheter in place in either soft tissue or a body cavity, and a medication infusion portion intended to deliver a medication to the infusion site. The two portions are formed independently and joined together in a side-by-side arrangement along at least the distal portion of the catheter.

The balloon fixation portion consists of a tube having an inflatable balloon at the distal end of the tube which can be inflated to a size sufficient to retain the catheter within the area of placement. A check valve assembly at the proximal end of the tube allows the balloon to be attached to an apparatus to inflate or deflate the balloon as required.

The medication infusion portion consists of a tube having one or more holes either at the distal end of the tube or along a distal portion of the tube for delivery of medication into the patient. At the proximal end of the tube attachment means, such as standard in the industry, is provided for attaching the infusion portion of the catheter to an infusion pump containing the medication to be delivered to the patient for the relief of pain.

During use, the distal end of the balloon catheter is placed at the desired site of infusion within the patient. The proximal end of the balloon fixation portion of the catheter is attached to an apparatus which is used to inflate the balloon in order to retain the catheter in place. The proximal end of the medication infusion portion of the catheter is attached to an infusion pump filled with the desired medication. The medication flows through the medication infusion portion of the catheter and out the holes at or near the distal end of the catheter thereby providing medication in the area of infusion. Medication typically provided comprises, but is not limited to, pain relief medication, antibiotics, chemotherapy or other treatment compositions, When infusion is complete, the balloon is deflated and the catheter is pulled from the body.

### BRIEF DESCRIPTION OF THE DRAWINGS

The above and other objects and advantages of the present invention will become apparent from the following description when read in conjunction with the accompanying drawings wherein:

FIG. 1 is a schematic representation of a first embodiment of a balloon catheter for providing flow out the distal end of the medication infusion portion of the catheter;

FIG. 2 is a schematic representation of a second embodiment of a balloon catheter for providing flow out of side holes near the distal end of the medication infusion portion of the catheter;

FIG. 3 is a cross-sectional view of a distal portion of a balloon catheter taken along line 3-3 of FIG. 2 showing the side-by-side attachment of the balloon fixation portion to the medication infusion portion of the catheter;

FIG. 4 is a schematic representations showing an over-the-needle peel-apart introducer placed near the ischial spine of the pelvis for use in inserting a balloon catheter into this surgical site for drug delivery following vaginal/pelvic floor repair.

FIG. 5 is a schematic representations showing the distal end of a balloon catheter incorporating features of the invention inserted through the lumen of the peel-apart introducer sheath with the balloon placed near the ischial spine of the pelvis.

FIG. 6 is a schematic representations showing the distal end of a balloon catheter incorporating features of the invention in place in the pelvic floor region with the balloon inflated near the ischial spine of the pelvis.

### DESCRIPTION OF THE INVENTION

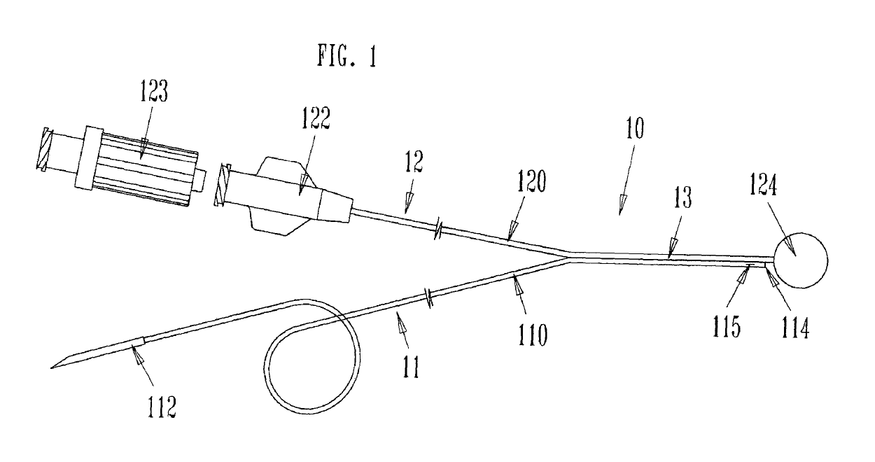

Referring to the drawings and particularly to FIG. 1, the preferred embodiment of a balloon catheter 10 for infusing a medication into the human body is illustrated. As indicated in FIG. 1, the balloon catheter 10 of this invention is comprised of two portions, a medication infusion portion 11 and a balloon fixation portion 12. A tubing length 13 of the medication infusion portion 11 and a balloon fixation portion 12 near the distal ends of both portions is bonded together in a side-by-side tubing configuration to form the balloon catheter 10. The medication infusion portion 11 is used to infuse a medication into the body. The balloon fixation portion 12 is used to fixate the catheter in the body at the infusion site for the duration of its use.

The medication infusion portion of the catheter 11 consists of a length of tubing 110 having an attachment means 112 for attaching the tubing to an infusion pump at the proximal end of the tubing, and an open end 114 at the distal end of the tubing for delivery of a medication to an infusion site.

In one embodiment, tubing 110 is formed from medical grade polyurethane having an outer diameter of 0.8 mm and a length of approximately 120 cm. This tubing has a very accurate and consistent internal diameter and an accurate length suitable for the tubing to be used as a flow restrictor. When connected to an infusion pump having a controlled pressure, the tubing restricts the flow rate of medication from the pump to a desired value, typically between 0.5 ml/hr to 10 ml/hr.

The attachment means 112 for connecting the tubing to the infusion pump will vary depending on the style of the infusion pump. In a preferred embodiment, the attachment means 112 consists of a stainless steel needle bonded to the proximal end of the tubing using an adhesive such as cyanoacrylate. During use, the needle is inserted through the elastomer septum of the infusion pump which provides medication under a controlled pressure. Other attachment means could include Luer™ connectors, Touhy Borst™ connectors, etc.

In this embodiment of the catheter, the distal end 114 of the tubing is open to allow the medication to flow out the end of the tube and into the body. Slit style infusion ports 115 may also be placed near the distal end of the tubing to allow flow from the side of the tubing in the event the end of the tubing becomes blocked. Typically, these slit style infusion ports are formed by penetrating the sharp tip of a blade through the tubing to form a cut parallel to the axial length of the tubing. The slit would typically having a length of approximately 0.8 mm. The slit port will open only when the pressure inside the tube, caused by a blockage, exceeds the ambient pressure at the site of infusion.

The balloon fixation portion of the catheter 12 consists of a length of tubing 120 having a second attachment means 122 at the proximal end for attaching the tubing to an apparatus used to fill and deflate the balloon, and an inflatable balloon 124 (depicted in the inflated state) at the distal end of the tubing. The balloon 124 is used to hold the catheter in place at an infusion site for delivery of the medication into the body.

In the first embodiment, tubing 120 is formed from medical grade polyurethane having an outer diameter of 1.0 mm and a length of approximately 40 cm. The length of this tubing is selected to enable the balloon filling and deflating apparatus, which will be later attached to the catheter, to be positioned away from the site where the catheter enters into the body.

The second attachment means 122 for attaching the tubing to an apparatus used to fill and deflate the balloon may vary depending on the options available to the manufacturer. In a preferred embodiment, a Luer™ connector 122 with a Luer-Lock™ input connection is attached to the proximal end of the tube using an adhesive such as cyanoacrylate. A syringe-activated check valve 123 is attached to this Luer connector using the luer-lock connection. Alternatively, the check valve 123 may be built into the attachment means and may be an integral part thereof.

During use, a syringe filled with air, saline, or some other substance, is attached to the proximal end of the check valve123. This causes the check valve to open and the balloon can be inflated or deflated using the syringe. When the syringe is removed from the check valve123, the balloon will remain inflated. At the end of the medication infusion period, the attached check valve 123 can be unscrewed from the Luer™ connector at the luer-lock connection and the balloon will deflate on its own.

An inflatable balloon 124 is attached to the distal end of the tubing 120 in order to fixate the catheter 10 at the infusion site. This diameter and style of the balloon 124 is selected based on the intended application of the balloon catheter 10. The balloon 124 may be attached to the tubing using a variety of methods including heat bonding, adhesive bonding, solvent bonding, etc. In a preferred embodiment, the balloon 124 is formed from medical grade polyurethane and has a spherical geometry. The diameter of the balloon 124 typically varies from 4 mm up to 2 cm and is attached to the tubing using heat bonding.

The tubing 110, 120 and balloon 124 used in the balloon catheter 10 can be formed from a variety of materials suitable for medical application. Tubing materials may include, but are not limited to, polyurethane, silicone elastomer, Teflon, Nylon, Pebax, etc. Balloon 124 materials may include polyurethane, silicone elastomers, polyisoprenes, etc. The tubing 110, 120 for each portion of the catheter does not need to be of the same material.

Both the medication infusion tube 110 and the balloon fixation tube 120 are attached along a length 13 of each tubing near their distal ends. In a preferred embodiment, the tubes 110, 120 are attached in a side-by-side manner for approximately 7 cm using a UV cure adhesive 34 suitable for polyurethane. Alternatively, the tubes 110, 120 can be attached after extrusion by heat, solvent, another adhesive, a melted polymer or they can be coextruded as a para-tube, in which case the shorter tube can be cut to length and the unwanted section of tubing peeled away from the longer tube.

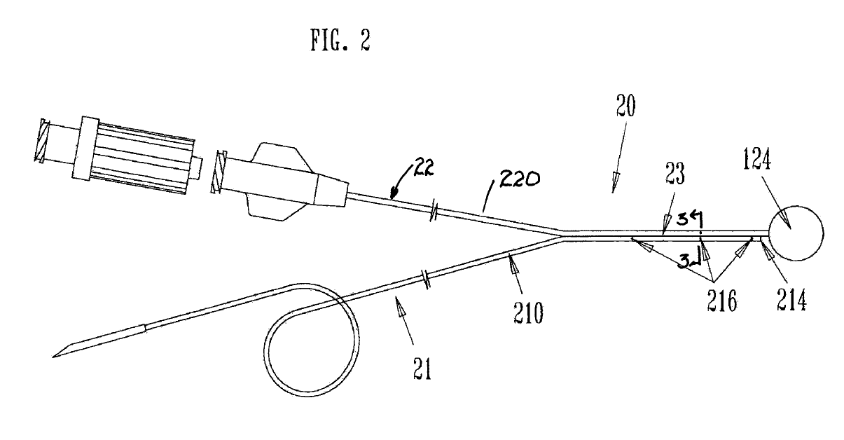

FIG. 2 shows an alternative embodiment of the balloon catheter 20. The balloon catheter 20 of this embodiment is similar, in most parts, to the balloon catheter 10 shown in FIG. 1. However, in the alternative embodiment, the medication infusion portion 22 of the catheter is different from the medication infusion portion 12 shown in the first embodiment of the balloon catheter 10. In particular, the distal end 214 of the tubing 210 is closed. Several hole style infusion ports 216 are placed near the distal end of the tubing 210 to allow the medication to be delivered along a length of the tubing rather than at the end of the tube.

One or more hole style infusion ports 216 may be placed along the distal length of the tubing 210 depending on the intended use of the catheter. Generally, all of the infusion ports would be contained in the bonded side-by-side portion 23 of the catheter. Typically, these hole style infusion ports 216 are formed by drilling or laser ablating the tubing to form a small opening. The size, style, and quantity of the ports are selected based on the technology used to fabricate the ports, the number of ports desired, their desired location along the distal length of the tubing 210, and the flow rate of the tubing 220.

In a preferred embodiment of a balloon catheter 20 having a flow rate of 4 ml/hr, several ports216 having a diameter of approximately 0.05 to 0.07 mm are drilled at 1 cm intervals along the catheter. In addition, multiple ports 216 are placed near the end of the catheter to ensure that there are sufficient openings so that all ports will flow except possibly a few near the distal end of the catheter.

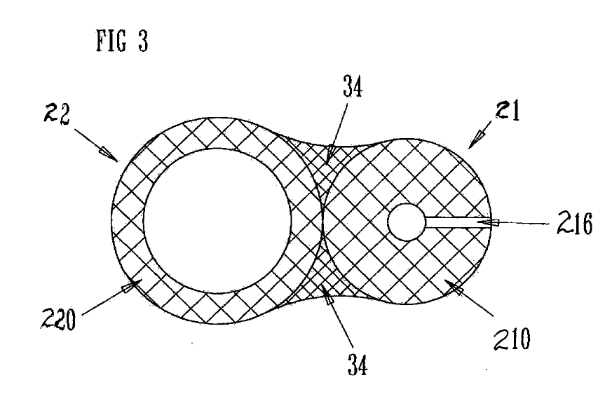

FIG. 3 shows a cross section taken along line 3-3 of FIG. 2 of the bonded side-by-side portion 23 of the balloon catheter 20 cut through an infusion port 216. In a preferred embodiment, the tubing 210 in the medication infusion portion 21 of the catheter 20 is bonded to the tubing 220 in the balloon fixation portion of the catheter 20 using a UV cure adhesive 34.

The diameter of tubing 220 in the balloon fixation portion 22 of the catheter is chosen so that the balloon can be quickly inflated and deflated. In a preferred embodiment, tubing 220 would have an outer diameter of 1.0 mm and an inner diameter of 0.7 mm. The diameter of tubing 210 in the medication infusion portion 21 of the catheter is chosen so that it can function as a flow restrictor and limit the flow rate of medication from the infusion pump into the patient. In a preferred embodiment for a balloon catheter 20 having a flow rate of 4 ml/hr, tubing 210 would have an outer diameter of 0.8 mm, an inner diameter of 0.18 mm, and a length of 120 cm.

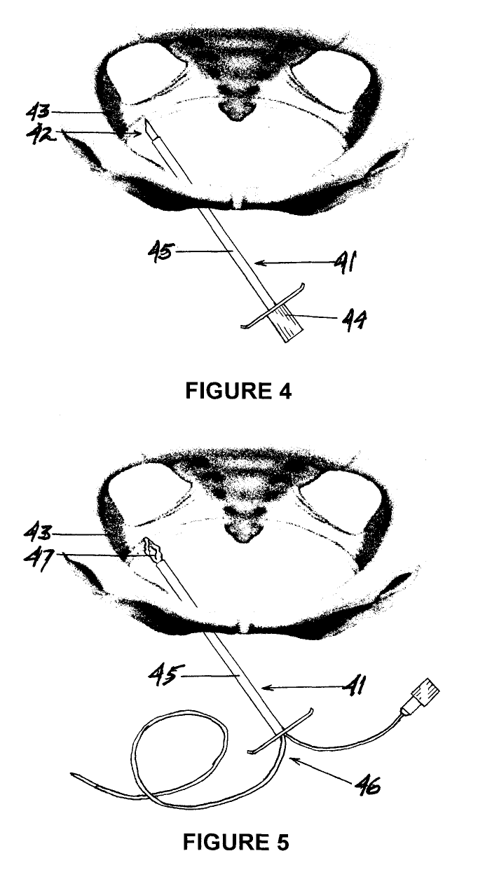

Referring to FIG. 4-6 there is disclosed a method of inserting, fixating, and removing a balloon fixated catheter in accordance with an embodiment of the invention at a site within the body. Following surgery for vaginal/pelvic floor repair, FIG. 4 shows an over-the-needle peel-apart introducer 41, which is well known in the art, inserted through the vaginal wall or floor tissue or through the abdomen so that the tip 42 of the introducer is close to the ischial spine 43 which is in the area of attachment of the surgical mesh used in the vaginal/pelvic floor repair. The introducer can be placed vaginally, transabdominally, or laparoscopically. Following placement of the introducer 41, the introducer needle portion 44 is removed, leaving only the introducer peel-apart sheath portion 45 in place.

In FIG. 5 the distal end of a balloon fixated catheter 46 has been slid down the lumen of the introducer peel-apart sheath portion 45 until the balloon 47 is near the ischial spine 43 which is close to the pudendal nerve in the surgical site. Following placement of the balloon fixated catheter 46, the introducer peel-apart sheath portion 45 is removed, leaving only the distal end of the balloon fixated catheter 46 in place.

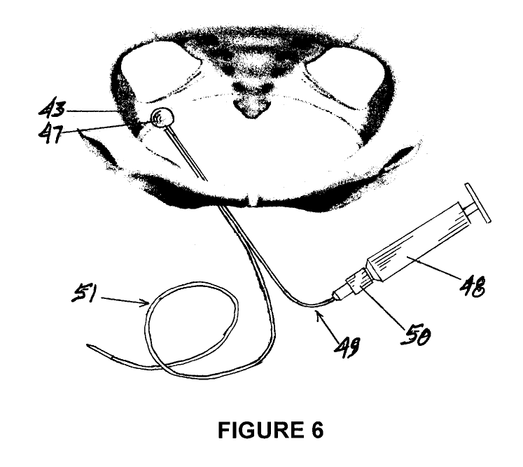

As shown in FIG. 6, a syringe 48 filled with air or water is attached to the proximal end of the balloon fixation portion 49 of the catheter. The balloon 47 at the distal end of the catheter is inflated until the distal end of the catheter is held gently in place at the surgical site. The syringe 48 is removed from the proximal end of the catheter leaving the balloon 47 inflated in the body. The proximal end of the medication infusion portion of the catheter 51 is attached to an infusion pump (not shown) and medication, for example for the relief of pain, is infused into the surgical site.

At the end of the infusion period, the check valve assembly 50 is removed from the Luer™ connector on the proximal end of the balloon fixation portion 49 of the catheter. This allows the balloon to deflate, and the catheter is pulled from the body.

Although this invention has been described in terms of certain preferred embodiments, other embodiments that are apparent to those of ordinary skill in the art are also within the scope of this invention. Accordingly, the scope of the invention is intended to be defined only by reference to the appended claims.

What is claimed is:

1. A catheter for infusion of a medication into the human body comprising:
a first portion for fixation of the catheter at an infusion site in the body comprising a tube having an inflatable balloon at its distal end and attachment means at its proximal end for connecting the tubing to an apparatus for filling and deflating the balloon;
a second portion for delivery of a medication to the infusion site in the body comprising a tube having attachment means at the proximal end of the tube for connecting the tube to an infusion pump, said infusion pump containing the medication to be infused, and one or more exit ports at the distal end of the tube for delivering the medication into the infusion site in the body;
said first and second portions provided as separate components joined together along a distal length of their respective tubes in a side-by-side manner.

2. The catheter of claim 1 wherein the distal end of the medication infusion portion of the catheter has an open end.

3. The catheter of claim 1 wherein the distal end of the medication infusion portion of the catheter has a closed end, said infusion portion having one or more holes spaced from the distal end of the tubing.

4. The catheter of claim 1 wherein the tubing forming the medication infusion portion of the catheter has an internal diameter and length selected so that pressurized medication fed to the tubing is delivered from the catheter at a flow rate of between 0.5 ml/hr and 10 ml/hr.

5. The catheter of claim 1 wherein a distal length of tubing comprising the medication infusion portion is joined to a distal length of tubing comprising the fixation portion in a side-by-side manner using an adhesive, heat, solvent, or a melted polymer.

6. The catheter of claim 6 wherein the joined distal length is about 7 cm.

7. The catheter of claim 6 wherein the tubing is polyurethane and adhesive is a UV cure adhesive compatible with polyurethane.

8. A method of infusing a medication into a patient for the relief of pain comprising:
providing a medication infusion catheter comprising a ballon fixation portion and a medication infusion portion, each portion provided as separate structures which are joined along at least a distal portion of their length following formation of said portions,
placing the balloon fixation portion of the catheter at a desired site of infusion within a patient, attaching the proximal end of the balloon fixation portion of the catheter to an apparatus for inflating the balloon, and inflating the balloon to retain the catheter at the site of infusion;
attaching the proximal end of the medication infusion portion of the catheter to an infusion pump filled with the desired medication and infusing said medication through the medication infusion portion of the catheter into the patient at the infusion site;
when infusion is complete, deflating the balloon and pulling the catheter from the body.
